(19) United States
(12) Patent Application Publication
(10) Pub. No.: US 2020/0352451 A1
(43) Pub. Date: Nov. 12, 2020

(54) Body Temperature Monitoring Pad and the System thereof

(71) Applicant: Digital Heat Technology Ltd., Hong Kong (CN)

(72) Inventor: Nam TSUI, Hong Kong (CN)

(21) Appl. No.: 16/404,781

(22) Filed: May 7, 2019

(51) Int. Cl. A61B 5/01; A61B 5/00

(52) U.S. Cl. CPC A61B 5/01; A61B 5/0022; A61B 5/0008; A61B 5/746; A61B 5/6833; A61B 5/6823; H01M 10/0525; A61B 5/0024; A61B 5/002; A61B 2562/0271; A61B 2562/164; A61B 2560/0214; A61B 5/6822

(57) ABSTRACT

A body temperature monitoring system for monitoring the body temperature continuously and providing alert in an event of detecting temperature irregularity is disclosed. The monitoring system includes a monitoring pad attachable to the human body for measuring the body temperature at various positions, and transmitting the collected data to the cloud system. The monitoring system further includes a cloud system having a database, and a mobile device having a transceiver, a processor, and a mobile application for configuring the monitoring system and receiving the alert. The database includes a device specific database and a clinical database, which facilitates the processor to identify a temperature irregularity when the measured body temperature is consistently outside an acceptable range of body temperature. The monitoring pad can be attached to the human body at various positions and accurately identify any temperature irregularity.

### FIELD OF THE INVENTION

The present invention relates to a body temperature monitoring device, and more particularly, to a body temperature monitoring pad attachable to the human body for measuring temperature, and a body temperature monitoring system having the device.

### BACKGROUND

The body temperature set-point is the level at which the body attempts to maintain for keeping the organism at optimum operating temperature. The body temperature reading normally fluctuates over the day and may be affected by a number of individual conditions. According to the American Medical Association, normal body temperature of an individual can range from approximately 97.8 F (36.5° C.) to 99 F (37.2° C.). Fever is an elevation in body temperature above the body temperature set-point, which is the result of the immune system response in the event of a medical condition, such as common cold, flu, gastroenteritis, bacterial infections, malaria, teething, medical side effects, post-vaccination effects, and other diseases. Meanwhile, an abnormally low body temperature of less than 95 F (35° C.) may cause a hypothermic state which can affect and impair the rate at which chemical reactions in the body take place and possible lead to respiratory or circulatory failure.

Fever is not a disease but a medical sign that a person may be sick, and is particularly common in young children. Many parents regularly monitor their children's body temperature as a mean for determining the health condition. When there is a higher body temperature, parents will tend to measure the body temperature more frequently and give their children fever-reducing medicine.

Thermometers are commonly used to measure oral, axillary (armpit), rectal, or eardrum temperatures. Taking the temperature with thermometer can confirm the diagnosis of a fever. Generally, a person having a body temperature greater than 100.4 F (38° C.) is considered to have a fever, whereas when the temperature is greater than 104 F (40° C.), the person is considered to be suffering from a high fever.

Patient with certain diseases, such as common cold, needs to have the body temperature measured regularly for monitoring the medical status. Typically, the body temperature is measured using thermometer by inserting the thermometer into the patient's mouth, ear, rectal, and the like. In Particular, children may not be able to measure their body temperature themselves. Therefore, the parents are required to perform the measurement for their children regularly even at night. However, such measurement activities may disturb the sleep of the patient, and may cause inconvenience to the parents.

There are some monitoring devices attachable to the human body for tracking the patient's body temperature over time. A continuous measurement can provide a vital statistic on temperature of the patient, which is specifically useful for preventing an occurrence of a high fever or a hypothermic state. This can minimize the risk of convulsions, hallucinations, or confusion of the patient as a result of the high fever. The problem with the conventional body temperature monitoring devices is the rigidness and inflexibility of the device, preventing the attachment of the device to the body at various positions. Furthermore, the device is usually required to be attached to a designated position, such as forehead, and the accuracy of the system for detecting temperature irregularity is compromised when the device is attached to other positions of the body.

Accordingly, there is a need in the art to have a body temperature monitoring device and system, which can continuously monitor the body temperature of a patient and provide alert in an event of detecting a temperature irregularity. In particular, the body temperature monitoring system having a clinical database and a device specific database for determining, in accordance with the attached position and other factors, whether the measured temperature is within the acceptable range of body temperature.

### SUMMARY OF THE INVENTION

It is an object of the present disclosure to provide a body temperature monitoring system for monitoring the body temperature continuously and providing alert in an event of detecting temperature irregularity.

In accordance with certain embodiments of the present disclosure, the monitoring pad attachable to and detachable from an individual for measuring a body temperature of the individual comprises a top layer comprising a protrusion and a covering sheet; a flexible circuit board comprising a temperature sensor, an analog-to-digital converter, a transmitting unit, and a flexible battery; a silicone sheet having an opening around a center of the silicone sheet; and a transparent sheet. The top layer and the silicone sheet are overlapped with each other and adhered together to form a cavity for interposing the flexible circuit board. The opening is positioned substantially away from all edges of the device for forming an insulating shield and reducing any influence to the temperature sensor by ambient temperature variations when the device is adhesively attached to the individual.

In accordance with a further aspect of the present disclosure, the flexible battery is a rechargeable lithium ceramic battery using a solid-state ceramic electrolyte. Alternatively, the flexible battery can also be a non-rechargeable thin-film battery.

In accordance with a further aspect of the present disclosure, the flexible circuit board further comprises a timer and memory cells. The timer periodically generates an enable signal to the temperature sensor to trigger the temperature sensor to sense the body temperature of the individual.

In accordance with a further aspect of the present disclosure, the temperature sensor is facing downwardly for measuring the body temperature of the individual to obtain a temperature data when the device is attached to the individual. In particular, the temperature sensor is an infrared temperature sensor or other non-contact type temperature sensors. Alternatively, the temperature sensor can be a contact type temperature sensor for achieving a higher measurement accuracy.

In accordance with yet a further aspect of the present disclosure, the temperature data comprises a single temperature reading, a set of temperature readings over time, a digital form of the temperature readings, or any combination thereof. The digital form of the temperature readings is obtainable by converting the set of temperature readings over time using the analog-to-digital converter.

In accordance with yet a further aspect of the present disclosure, the temperature data comprises an identification code or a time parameter.

In accordance with a further aspect of the present disclosure, the protrusion and the flexible circuit board are flexible and capable of bending by a degree that allows the device to adhere stably on a skin surface of the individual.

In accordance with a further aspect of the present disclosure, the transparent sheet is a skin-friendly layer provided with an adhesive at a bottom exposed surface for attaching to the individual.

In accordance with another aspect of the present disclosure, a monitoring system, for continuously monitoring a body temperature at a plurality of attached positions of an individual and providing an alert when a temperature irregularity is identified, comprises a cloud system comprising a database; a mobile device comprising a processor, a transceiver, and a mobile application for configuring the system and receiving the alert; and a monitoring pad of claim 1 configured to measure the body temperature at the plurality of attached positions, generate a temperature data, and transmit the temperature data to the mobile device. The database comprises a device specific database and a clinical database. The processor is configured to generate the alert to indicate the temperature irregularity when the temperature data is consistently outside an acceptable range of body temperature.

In accordance with a further aspect of the present disclosure, the acceptable range of body temperature for the attached position is provided by the clinical database according to the individual's age, gender, race, weight, and other demographic data.

In accordance with a further aspect of the present disclosure, the mobile application comprises a configuration unit for collecting information in relation to the attached position and the individual's age, gender, race, weight, and other demographic data.

In accordance with a further aspect of the present disclosure, the monitoring pad is flexible and capable of bending by a degree that allows the device to adhere stably on a skin surface of the individual.

In accordance with a further aspect of the present disclosure, the plurality of attached positions are a frontal region, a sternal region, an epigastric or an umbilical region, a dorsum region, a back neck region, and an armpit region.

In accordance with a further aspect of the present disclosure, the device specific database is used to store past records of the temperature data for improving the accuracy of analysis.

In accordance with a further aspect of the present disclosure, the temperature data is transmitted to the cloud system by Wi-Fi, Wireless Body Area Network (WBAN), Bluetooth, Zigbee, or a combination thereof.

In accordance with a further aspect of the present disclosure, the alert triggers a notification or a pop-up message in the mobile application, an automatically generated e-mail, a short message service (SMS), or any combination thereof.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Other aspects of the present invention are disclosed as illustrated by the embodiments hereinafter.

### BRIEF DESCRIPTION OF THE DRAWINGS

The appended drawings, where like reference numerals refer to identical or functionally similar elements, contain figures of certain embodiments to further illustrate and clarify various aspects, advantages and features of the body temperature monitoring pad and the system thereof as disclosed herein. It will be appreciated that these drawings and graphs depict only certain embodiments of the invention and are not intended to limit its scope. The body temperature monitoring pad and the system thereof as disclosed herein will be described and explained with additional specificity and detail through the use of the accompanying drawings in which:

FIG. 1A depicts a monitoring system utilizing the monitoring pad in accordance with an exemplary embodiment of the present disclosure.

FIG. 1B depicts an alternative monitoring system utilizing the monitoring pad in accordance with an exemplary embodiment of the present disclosure.

FIG. 2 depicts five different ways of attaching the monitoring pad to a child in accordance with an exemplary embodiment of the present disclosure.

FIG. 3A depicts the bottom view of the monitoring pad in accordance with an exemplary embodiment of the present disclosure.

FIG. 3B depicts the top view of the monitoring pad in accordance with an exemplary embodiment of the present disclosure.

FIG. 3C depicts the side view of the monitoring pad in accordance with an exemplary embodiment of the present disclosure.

FIG. 3D depicts the front view of the monitoring pad in accordance with an exemplary embodiment of the present disclosure.

FIG. 4 is an exploded perspective view of the monitoring pad of FIGS. 3A-3D.

FIG. 5A is a block diagram of the monitoring system of FIG. 1A.

FIG. 5B is a block diagram of the alternative monitoring system of FIG. 1B.

Skilled artisans will appreciate that elements in the figures are illustrated for simplicity and clarity and have not necessarily been depicted to scale.

### DETAILED DESCRIPTION

The present disclosure generally relates to a device for monitoring the body temperature. More specifically, but without limitation, the present disclosure relates to a body temperature monitoring pad attachable to the human body for measuring temperature continuously, and a body temperature monitoring system having the device that can provide alert in an event of detecting temperature irregularity.

The following detailed description, the body temperature monitoring pad is merely exemplary in nature and is not intended to limit the disclosure or its application and/or uses. It should be appreciated that a vast number of variations exist. The detailed description will enable those of ordinary skill in the art to implement an exemplary embodiment of the present disclosure without undue experimentation, and it is understood that various changes or modifications may be made in the function and arrangement of the device of operation described in the exemplary embodiment without departing from the scope of the present disclosure as set forth in the appended claims.

Aspects of the present invention may be embodied as a system, method or computer program. The present invention may be implemented by hardware, software, firmware, middleware, microcode, hardware description languages, or any combination thereof. When implemented in software, firmware, middleware or microcode, the program code or code segments to perform the necessary tasks may be stored in a machine-readable medium such as a non-transitory storage medium. A processor(s) may perform the necessary tasks. A code segment may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

The term “cloud” is construed and interpreted in the sense of cloud computing or, synonymously, distributed computing over a network unless otherwise specified. “A server” as used herein is interpreted in the sense of computing. The one or more “database” may be, for example, electrical circuits, hard disks and/or other solid-state disks for storing data. Generally, a server is equipped with one or more processors for executing program instructions, and/or one or more storages for storing data. The server may be a standalone computing server or a distributed server in the cloud.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The use of any and all examples, or exemplary language (e.g., “such as”) provided herein, is intended merely to illuminate the invention better and does not pose a limitation on the scope of the invention unless otherwise claimed. No language in the specification should be construed as indicating any non-claimed element as essential to the practice of the invention.

Terms such as “upper”, “lower”, “top”, “bottom”, “front”, “rear”, and variations thereof herein are used for ease of description to explain the positioning of one element relative to a second element, and are not intended to be limiting to a specific orientation or position. Terms such as “first”, “second”, and variations thereof herein are used to describe various elements, regions, sections, etc. and are not intended to be limiting.

Terms such as “connected”, “in communication with”, and variations thereof herein are used broadly and encompass direct and indirect connections, and communication; and are not restricted to electrical, physical or mechanical attachments and connections.

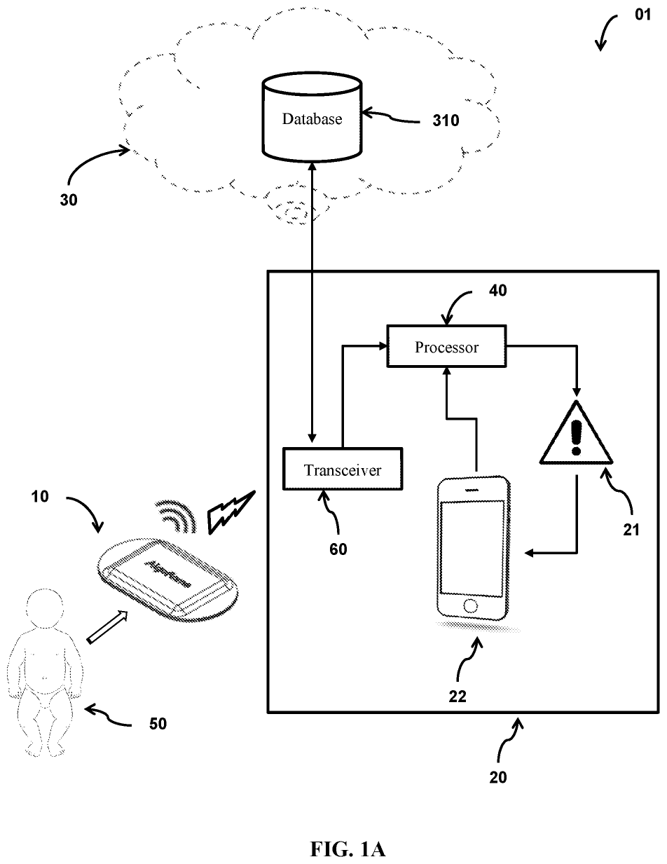

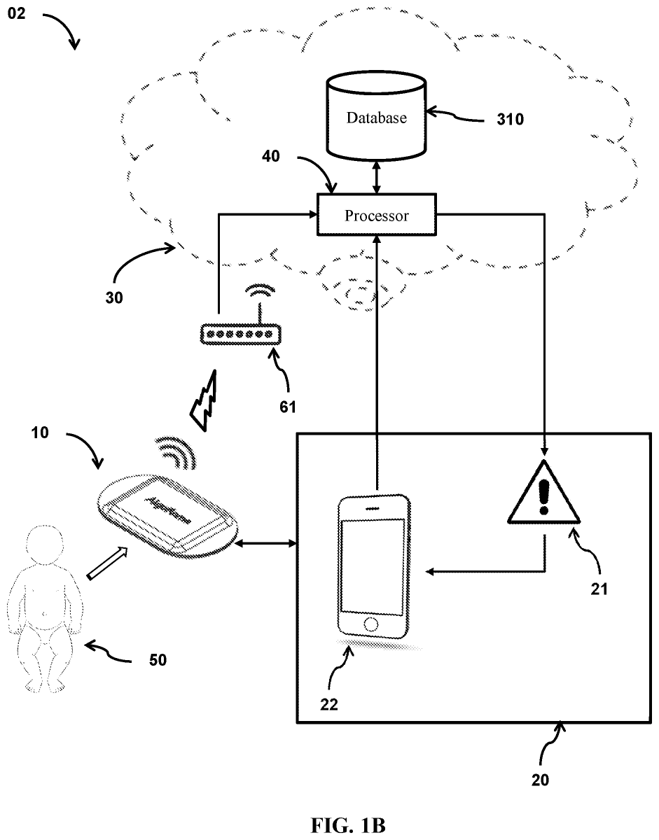

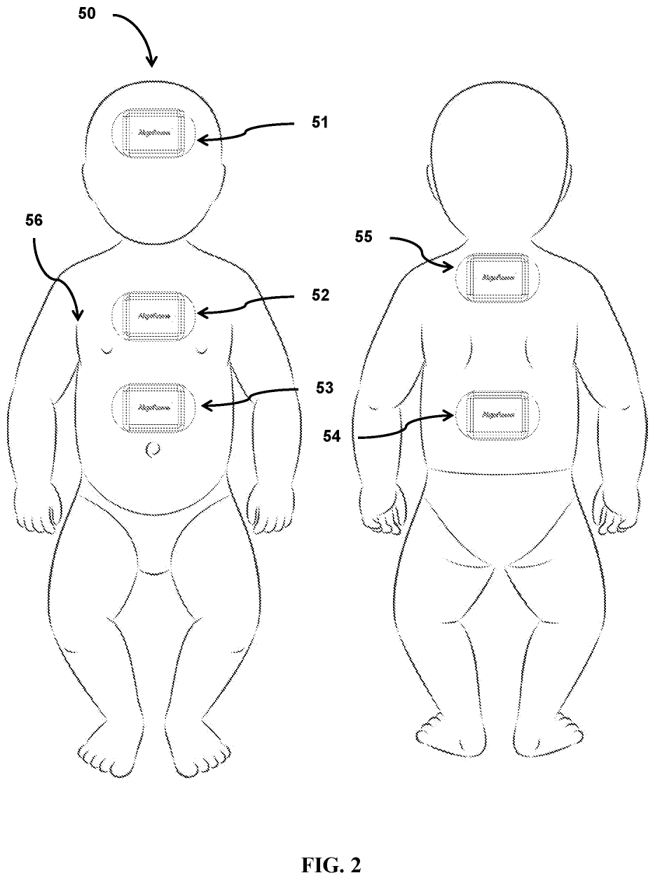

Referring to FIG. 1A, a monitoring system 01 is described in details. The monitoring pad 10 is a device, which is attachable to and detachable from an individual 50, for measuring the body temperature of the individual 50. Although an infant is illustrated in FIG. 1A (also in FIG. 1B and FIG. 2), the monitoring pad 10 and the monitoring system 01 thereof as described herein are applicable to both children and adults. The monitoring pad 10 acquires body temperature at the skin surface of an attached position, processes the acquired body temperature into temperature data, and transmitting the temperature data to a mobile device 20 or other electronic devices for further processing. The temperature data may include a single temperature reading, a set of temperature readings over time, a digital form of the temperature readings, or any combination thereof. In certain embodiment, an identification code or a time parameter is also included in the temperature data and transmitted to the mobile device 20. The monitoring pad 10 is paired to or otherwise connected to the mobile device 20 through the transceiver 60 using wireless transmission. The transmission can be implemented by Wi-Fi, and it should be readily understood that any other wireless communication method of data transmission may be employed, including Wireless Body Area Network (WBAN), Bluetooth, Zigbee, or a combination thereof. Caching techniques may also be adopted to guarantee smooth data transmission. The temperature data is transmitted to and received by a transceiver 60, which is internal to the mobile device 20. The transceiver 60 can include a cellular transceiver, a Wi-Fi transceiver, a Bluetooth transceiver, a data port, and an antenna port. As discussed in more detail below, these various components in the mobile device 20 can be controlled by the processor 40 to obtain and transmit data such as temperature data, configuration information, and acceptable range of temperature.

The processor 40 is configured to execute a method to receive and analyze the temperature data for determining the body temperature regularity. The processor 40 receives configuration information in relation to the attached position of the monitoring pad 10 from the mobile application 22, which is downloaded and stored in the memory cells of the mobile phone 20, and identifies an acceptable range of temperature of the individual from the database 310, which is obtainable from a cloud system 30 through the transceiver 60. The configuration information from the mobile application 22 determines the appropriate set of normal body temperature in the database 310 to be used for analysis. Having knowledge of the normal body temperature, the processor 40 can identify any temperature irregularity. The temperature irregularity can be an elevation or a drop in body temperature beyond the acceptable range of temperature.

In case a temperature irregularity is identified by the processor 40, an alert 21 is generated and provided to the mobile application 22 to indicate such temperature irregularity. The processor 40 may also be provided with the ability to recognize an increasing trend in temperature data and make prediction on a temperature irregularity. The alert 21 can trigger an alert system 220 and provide a notification in the form of a pop-up message in the mobile application 22, an automatically generated e-mail, or a short message service (SMS).

Now referring to FIG. 1B, an alternative monitoring system 02 is described in details. With the same monitoring pad 10, body temperature is acquired at the skin surface of an attached position, processes the acquired body temperature into temperature data, and transmitting the temperature data to the cloud system 30 for further processing. The transmission is implemented by Wi-Fi, and it should be readily understood that any other wireless communication method of data transmission may be employed, including Wireless Body Area Network (WBAN), Bluetooth, Zigbee, or a combination thereof. Caching techniques may also be adopted to guarantee smooth data transmission. The temperature data is transmitted to and received by a receiver 61, which can be a transceiver device such as a wireless modem, a router, a Zigbee receiver, or a Bluetooth device, and transmitted to a designated server in the cloud system 30 for further processing. The monitoring pad 10 may also be paired to or otherwise connected to a mobile device 20 using wireless transmission.

The processor 40 is configured to execute a method to receive and analyze the temperature data for determining the body temperature regularity of the individual 50. The processor 40 also receives configuration information from the mobile application 22 in relation to the attached position of the monitoring pad 10 and identifies an acceptable range of temperature of the individual from the database 310. It should also be understood that the configuration information from the mobile application 22 can be transmitted to the processor 40 directly through network, or transmitted to the monitoring pad 10 which in turn further transmitted to the processor 40 together with the temperature data. Having knowledge of the normal body temperature, the processor 40 can identify any temperature irregularity.

In the alternative monitoring system 02, the alert 21 can be made through a computer network or by wireless transmission to the mobile device 20 or other electronic devices, trigger an alert system 220 and provide a notification in the form of a pop-up message in the mobile application 22, an automatically generated e-mail, or a SMS.

FIG. 2 illustrates five different ways of attaching the monitoring pad 10 on an individual 50. In general, the attached positions are those positions in the trunk and the head of the individual 50 in which the monitoring pad 10 can adhere comfortably thereto. In particular, the preferred attached positions are frontal region 51, sternal region 52, epigastric or umbilical region 53, dorsum region 54, back neck region 55, and armpit region 56. Apart from those positions indicated in FIG. 2, the monitoring pad 10 can also be attached to anywhere in the vicinity. The true core body temperature is the temperature of the arterial blood flow from the heart, which is most accurately measured at the center of the heart. The true core body temperature provides important information on the temperature set-point for determining whether the individual 50 is having temperature irregularity. Generally, the limbs cannot provide a good correlation with the core temperature at the skin surface, while the trunk and neck may exhibit excellent correlation to core temperature at the skin surface. However, in order to ensure an accurate data acquisition, temperature insulation from the environment is implemented in the present disclosure, which will be more fully described herein.

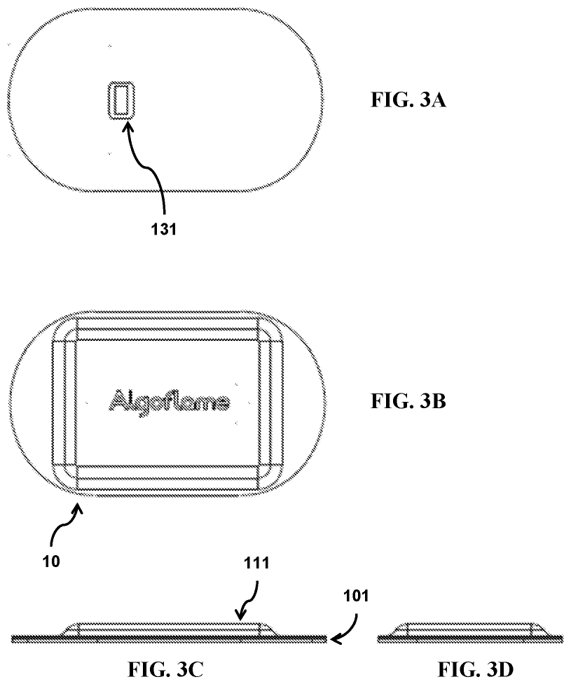

FIGS. 3A-3D show various views of the monitoring pad 10 in accordance with certain embodiments of the present disclosure. The monitoring pad 10 may be in an oval shape, a rectangular shape, a circular shape, a shape similar to an adhesive bandage, or any other desirable shape. FIG. 3A shows a bottom view; FIG. 3B shows a top view; FIG. 3C shows a side view; and FIG. 3D shows a front view. The monitoring pad 10 may include a plurality of flexible sheets 101 and a rectangular shaped protrusion 111 overlapped with and attached above the flexible sheets 101. The protrusion 111 has a longitudinal width substantially less than the longitudinal width of the flexible sheets 101, and a transverse height equal to or slightly less than the transverse height of the flexible sheets 101. In certain embodiments, the size of the monitoring pad 10 can be 40.4 mm×69 mm. Advantageous, the protrusion 111, and the circuit board within (not shown in FIG. 3A-3D) are flexible and capable of bending by a degree that allows the monitoring pad 10 to adhere stably on the skin surface of the individual 50.

On the bottom of the monitoring pad 10, there is provided an opening 131 with a temperature sensor 121 embedded within and facing downwardly for measuring the body temperature when the monitoring pad 10 is attached to the individual 50. The temperature sensor 121 may be a non-contact type temperature sensor or a contact type temperature sensor. For the avoidance of doubt, the opening 131 is a hole on at least one of the flexible sheets 101, and the bottommost layer of the flexible sheets 101, which is preferred to be transparent, may not have a hole or a gap of any kind physically. The position and dimension of the opening 131 is not necessary be exactly the same as indicated in FIG. 3A. The opening 131 can have a circular shape, an oval shape, or any other desirable shape. The opening 131 is positioned substantially away from all the edges of the monitoring pad 10, such that when the monitoring pad 10 is adhesively attached to the skin, the area between the edge of the monitoring pad 10 and the opening 131 forms an insulating shield for reducing any influence to the non-contact type temperature sensor 121 by any ambient temperature variations.

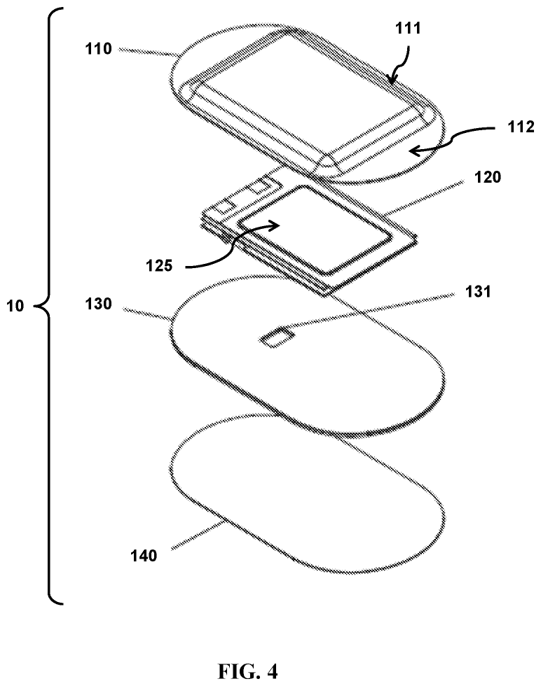

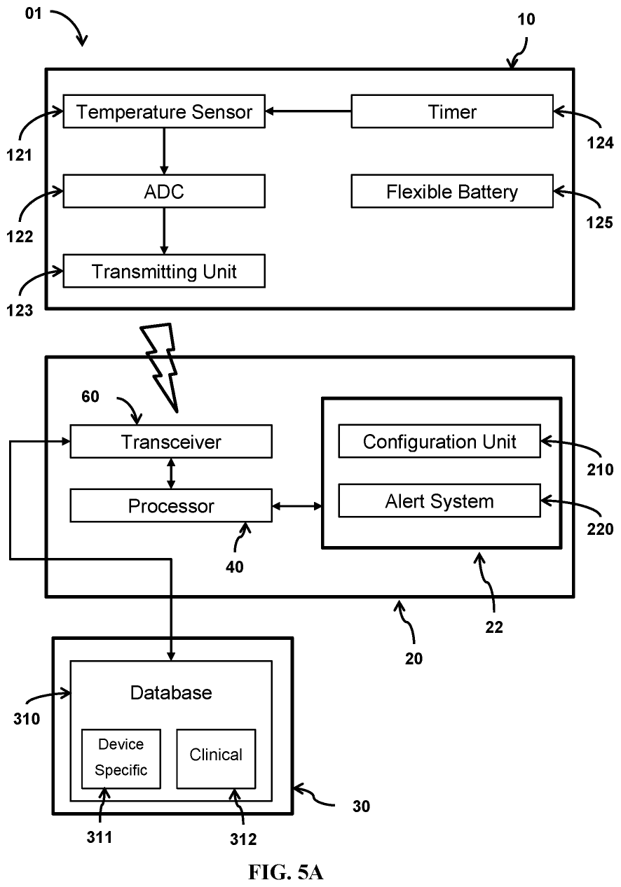

FIG. 4 shows an exploded perspective view of the monitoring pad 10. The monitoring pad 10 comprises a top layer 110, a flexible circuit board 120, a silicone sheet 130, and a transparent sheet 140. The top layer 110 comprises a protrusion 111 and a covering sheet 112, which is a flat and soft flexible layer of woven fabric, plastic (PVC, polyethylene, or polyurethane), or other material. The top layer 110 and the silicone sheet 130 are overlapped with each other and adhered together to form a cavity for interposing the flexible circuit board 120 therein. The flexible circuit board 120 is a stretchable and durable film with at least the following components mounted thereon, including, a temperature sensor 121, an analog-to-digital converter 122, a transmitting unit 123, and a flexible battery 125. A timer 124 (e.g.: crystal oscillator, clock signal generator) and memory cells may also be mounted on the flexible circuit board 120. The flexible battery 125 is a rechargeable lithium ceramic battery using solid-state ceramic electrolyte. The lithium ceramic battery has the advantage of good thermal stability without thermal runaway. Even when the lithium ceramic battery is penetrated through or damaged by folding, there is still no risk of fire or explosion. Alternatively, the flexible battery 125 can also be other chargeable or non-rechargeable thin-film battery, such as zinc manganese-dioxide battery or lithium polymer battery.

The silicone sheet 130 is a flexible insulating layer with an opening 131 around the center of the silicone sheet 130 which is sufficiently away from all the edges of the monitoring pad 10. The silicone sheet 130 is a good temperature insulator and the ambient temperature may not cause impact to the readings of the temperature sensor 121, in particular, when the monitoring pad 10 is readily attached to the skin for sufficiently long period of time (at least 1 minute), the insulation effect of the silicone sheet 130 is more noticeable.

The transparent sheet 140 is a skin-friendly layer, which is provided with an adhesive at the bottom exposed surface so that the monitoring pad 10 is attachable to and detachable from the skin of the individual 50.

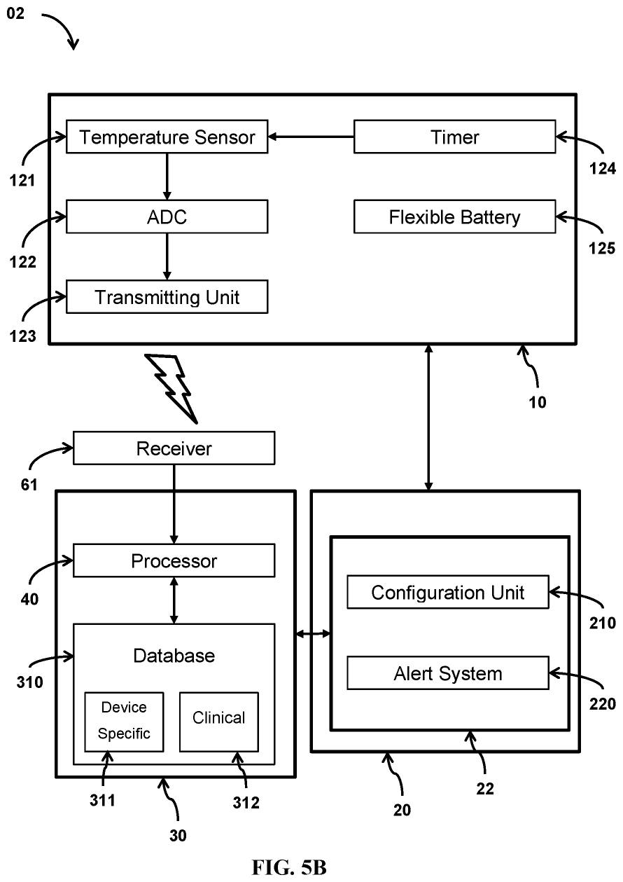

FIG. 5A is a block diagram of the monitoring system 01 having the monitoring pad 10. The monitoring system 01 includes a monitoring pad 10 attached to an individual 50 to sense the body temperature, a cloud system 30 having a database 313, a mobile device 20 having a transceiver 60, a processor 40 for determining the condition of the individual 50, and a mobile application 22 for configuring the monitoring system 01 and receiving alert 21. The operation of the monitoring system 01 according to a certain embodiment is described herein.

The monitoring pad 10 comprises a temperature sensor 121, an analog-to-digital converter 122, a transmitting unit 123, a timer 124, and a flexible battery 125. The timer 124 periodically generates enable signal to the temperature sensor 121 to trigger the temperature sensor 121 to sense the body temperature of the individual 50. The temperature sensor 121 is an infrared temperature sensor, or other non-contact type temperature sensors. Alternatively, the temperature sensor 121 can also be a thermocouple, a resistance temperature detector, a thermistor, or other contact type temperature sensors, which can achieve a higher measurement accuracy. The sensed body temperature can be a single temperature reading or a set of temperature readings over time, which may be converted to digital form using an analog-to-digital converter 122 and be processed to obtain temperature data. The temperature data is then transmitted to the mobile device 20 by the transmitting unit 123 by wireless technologies such as Wi-Fi, Wireless Body Area Network (WBAN), Bluetooth, Zigbee, or a combination thereof.

As the monitoring pad 10 can be attached to different positions of the individual 50, the monitoring system 01 requires at least the information on the attached position for determining whether the body temperature is within an acceptable range. Furthermore, individuals having similar demographics may have correlated trend of body temperature based on other factors such as age, gender, race, and weight. The variation is small and manageable for children, particularly the infants and young toddlers. A configuration unit 210 in the mobile application 22 allows the filling in of the preceding information and factors regarding the individual 50, which is then transmitted to the processor 40.

The monitoring pad 10 is paired to or otherwise connected to a mobile device 20 through the transceiver 60 using wireless transmission. The transmission is implemented by Wi-Fi, and it should be readily understood that any other wireless communication method of data transmission may be employed, including Wireless Body Area Network (WBAN), Bluetooth, Zigbee, or a combination thereof.

The cloud system 30 comprises a database 310, which includes a device specific database 311 and a clinical database 312. In certain embodiments, the mobile device 20 is configured to download sets of normal body temperature from the database 310 and store the data in the one or more memory cells in the mobile device 20.

The device specific database 311 is a record of the temperature data for a particular monitoring pad 10. The processor 40 retrieves past records of the temperature data for the attached position from the device specific database 312 for that particular monitoring pad 10 and makes comparison. The newly acquired temperature data can also be stored in the device specific database 312 to enrich the context and improve the accuracy for future analysis.

The clinical database 312 is a predefined and regularly updated database having clinical records of the acceptable range of body temperature of various attached positions from individuals sorted by their age, gender, race, weight, and other demographic data. Individuals of similar characteristics may have similar body temperature measurement. These demographic data can improve the accuracy of the monitoring system 01 and serve as an additional input for determining the condition of the individual. The processor 40 retrieves clinical records of the temperature data for the attached position from individual with similar demographic data from the clinical database 312 and makes comparison.

When the processor 40 determines that the received temperature data is consistently outside the acceptable range of body temperature for a period of time, the processor 40 will identify this as a temperature irregularity and generate an alert 21 to the mobile application 22 to indicate such temperature irregularity. The device specific database 311 and the clinical database 312 can facilitate the processor 40 to determine a more accurate acceptable range of body temperature for improving the accuracy of the analysis. The alert 21 can trigger an alert system 220 and provide a notification or a pop-up message in the mobile application 22, an automatically generated e-mail, a SMS, or any combination thereof.

FIG. 5B is a block diagram of an alternative monitoring system 02 having the monitoring pad 10. The monitoring system 01 includes a monitoring pad 10 attached to an individual 50 to sense the body temperature, a cloud system 30 having a database 310 and a processor 40 for determining the condition of the individual 50, and a mobile application 22 for configuring the alternative monitoring system 02 and receiving alert 21.

With the same monitoring pad 10 as the monitoring system 01, the temperature data is transmitted to the cloud system 30 by the transmitting unit 123 through the receiver 61 using wireless technologies such as Wi-Fi, Wireless Body Area Network (WBAN), Bluetooth, Zigbee, or a combination thereof. The monitoring pad 10 may also be paired to or otherwise connected to a mobile device 20 using wireless transmission.

The cloud system 30 comprises a processor 40 and a database 310, which includes a device specific database 311 and a clinical database 312. When the processor 40 determines that the received temperature data is consistently outside the acceptable range of body temperature for a period of time, the processor 40 will identify this as a temperature irregularity and generate an alert 21. The alert 21 can be made through a computer network or by wireless transmission to the mobile device 20 or other electronic devices, trigger an alert system 220, and provide a notification or a pop-up message in the mobile application 22, an automatically generated e-mail, a SMS, or any combination thereof, for indicating such temperature irregularity.

The present disclosure may be embodied in other specific forms without departing from the spirit or essential characteristics thereof. The present embodiment is therefore to be considered in all respects as illustrative and not restrictive. The scope of the disclosure is indicated by the appended claims rather than by the foregoing description, and all changes that come within the meaning and range of equivalency of the claims are therefore intended to be embraced therein.

What is claimed is:

1. A monitoring pad attachable to and detachable from an individual for measuring a body temperature of the individual, the device comprising:
a top layer comprising a protrusion and a covering sheet;
a flexible circuit board comprising a temperature sensor, an analog-to-digital converter, a transmitting unit, and a flexible battery;
a silicone sheet having an opening around a center of the silicone sheet; and
a transparent sheet;
wherein:
the top layer and the silicone sheet are overlapped with each other and adhered together to form a cavity for interposing the flexible circuit board; and
the opening is positioned substantially away from all edges of the device for forming an insulating shield and reducing any influence to the temperature sensor by ambient temperature variations when the device is adhesively attached to the individual.

2. The device of claim 1, wherein the flexible battery is a rechargeable lithium ceramic battery using a solid-state ceramic electrolyte.

3. The device of claim 1, wherein the flexible battery is a non-rechargeable thin-film battery.

4. The device of claim 1, wherein the flexible circuit board further comprises a timer and memory cells, such that the timer can periodically generate an enable signal to the temperature sensor to trigger the temperature sensor to sense the body temperature of the individual.

5. The device of claim 1, wherein the temperature sensor is an infrared temperature sensor or other non-contact type temperature sensors, which is facing downwardly for measuring the body temperature of the individual to obtain a temperature data when the device is attached to the individual.

6. The device of claim 5, wherein the temperature data comprises a single temperature reading, a set of temperature readings over time, a digital form of the temperature readings, or any combination thereof.

7. The device of claim 6, wherein the digital form of the temperature readings is obtainable by converting the set of temperature readings over time using the analog-to-digital converter.

8. The device of claim 6, wherein the temperature data further comprises an identification code or a time parameter.

9. The device of claim 1, wherein the protrusion and the flexible circuit board are flexible and capable of bending by a degree that allows the device to adhere stably on a skin surface of the individual.

10. The device of claim 1, wherein the transparent sheet is a skin-friendly layer provided with an adhesive at a bottom exposed surface for attaching to the individual.

11. A monitoring pad attachable to and detachable from an individual for measuring a body temperature of the individual, the device comprising:
a top layer comprising a protrusion and a covering sheet;
a flexible circuit board comprising a temperature sensor, an analog-to-digital converter, a transmitting unit, and a flexible battery;
a silicone sheet having an opening around a center of the silicone sheet; and
a transparent sheet;
wherein:
the top layer and the silicone sheet are overlapped with each other and adhered together to form a cavity for interposing the flexible circuit board; and
the temperature sensor is a contact type temperature sensor for achieving a higher measurement accuracy.

12. A monitoring system for continuously monitoring a body temperature at a plurality of attached positions of an individual and providing an alert when a temperature irregularity is identified, the system comprising:
a cloud system comprising a database;
a mobile device comprising a processor, a transceiver, and a mobile application for configuring the system and receiving the alert; and
a monitoring pad of claim 1 configured to measure the body temperature at the plurality of attached positions, generate a temperature data, and transmit the temperature data to the mobile device;
wherein
the database comprises a device specific database and a clinical database; and
the processor is configured to generate the alert to indicate the temperature irregularity when the temperature data is consistently outside an acceptable range of body temperature.

13. The system of claim 12, wherein the acceptable range of body temperature for the attached position is provided by the clinical database according to the individual's age, gender, race, weight, and other demographic data.

14. The system of claim 12, wherein mobile application comprises a configuration unit for collecting information in relation to the attached position and the individual's age, gender, race, weight, and other demographic data.

15. The system of claim 12, wherein the monitoring pad is flexible and capable of bending by a degree that allows the device to adhere stably on a skin surface of the individual.

16. The system of claim 12, wherein the plurality of attached positions are a frontal region, a sternal region, an epigastric or an umbilical region, a dorsum region, a back neck region, and an armpit region.

17. The system of claim 12, wherein the device specific database is used to store past records of the temperature data for improving the accuracy of analysis.

18. The system of claim 12, wherein the temperature data is transmitted to the mobile device by Wi-Fi, Wireless Body Area Network (WBAN), Bluetooth, Zigbee, or a combination thereof.

19. The system of claim 12, wherein the alert triggers a notification or a pop-up message in the mobile application, an automatically generated e-mail, a short message service (SMS), or any combination thereof.

20. A monitoring system for continuously monitoring a body temperature at a plurality of attached positions of an individual and providing an alert when a temperature irregularity is identified, the system comprising:
a cloud system comprising a processor and a database;
a mobile device comprising a mobile application for configuring the system and receiving the alert; and
a monitoring pad of claim 1 configured to measure the body temperature at the plurality of attached positions, generate a temperature data, and transmit the temperature data to the cloud system;
wherein
the database comprises a device specific database and a clinical database; and
the processor is configured to generate the alert to indicate the temperature irregularity when the temperature data is consistently outside an acceptable range of body temperature.
